(19) United States
(12) Patent Application Publication
(10) Pub. No.: US 2016/0279658 A1
(43) Pub. Date: Sep. 29, 2016

(54) ATOMIZING APPARATUS FOR DEVELOPMENT OF BIOLOGICAL TRACE EVIDENCE

(71) Applicants: Huang-Te Li, Hsinchu City (TW); ARVIN Bio-Medical Devices Co., Ltd., Hsinchu City (TW)

(72) Inventors: HUANG-TE LI, Hsinchu City (TW); CHENG-LUNG LEE, Hsinchu City (TW); CHIA-CHING CHOU, Hsinchu City (TW); WEI-CHEN CHEN, Hsinchu City (TW)

(21) Appl. No.: 14/665,779

(22) Filed: Mar. 23, 2015

(51) Int. Cl. B05B 17/06; G01N 33/58

(52) U.S. Cl. CPC B05B 17/0615; G01N 33/582; B05B 17/0676

(57) ABSTRACT

An atomizing apparatus for development of biological trace evidence comprises a container which has an opening and a housing space communicating with the opening, a trace evidence developing solution held in the housing space to develop a biological trace evidence and an atomizer located in the opening. The atomizer includes a piezoelectric vibration element in contact with the trace evidence developing solution. The piezoelectric vibration element can shake the trace evidence developing solution via high frequency vibration to form a plurality of small liquid particles to be discharged outside the container. The small liquid particles of the trace evidence developing solution pass through the opening and spray directly on the biological trace evidence to generate an oxidized reduction reaction therewith to produce colored substance to reveal the biological trace evidence in a visible fashion.

### FIELD OF THE INVENTION

The present invention relates to an apparatus for development of biological trace evidence and particularly to an atomizing apparatus adopted for use to develop biological trace evidence.

### BACKGROUND OF THE INVENTION

With development and advance of scientific technology and instruments now a biological trace evidence is widely used in the field of criminal identification to become one of acceptable object evidence. A biological trace evidence broadly means a unique biological characteristic of an individual entity that can be used to differentiate differences of each individual entity and identify the identity thereof, such as fingerprint, blood, hairs, body fluid and the like. Among them the fingerprint is the most commonly used biological trace evidence in the criminal identification.

Take the fingerprint as an example, the undulate traces of the skin on the finger pad at a human being's fingertip are different for each person, hence have an unique characteristic that can be used in personal identification, such as in a personal registration system, immigration identification, seal substitute, crime investigation and the like.

When a finger is in contact with an object it usually leaves a fingerprint on the object. The fingerprint contains sweat secreted by skin sweat glands. It is composed mostly of water, also includes protein, organic compounds, lipid and urea. This is why the fingerprint can be used as an investigation tool in the crime investigation. However, the fingerprint marked on the object is hardly visible by people's naked eyes, hence many types of methods to reveal the fingerprint and make it visible have been developed. Among them the mostly adopted methods are powder development method, smoke fumigation method, and Ninhydrin method.

The powder development method was adopted earlier. It adopts a principle of brushing silver powder or magnetic powder via a brush to adsorb to an object which has a fingerprint trace marked thereon. The silver powder or the magnetic powder adsorbs to the water by electrical static charges to reveal the image of the fingerprint so that it becomes visible. However, during brushing of the silver powder or the magnetic powder the brush directly touches the fingerprint and has the risk of destroying the fingerprint. Moreover, the water diminishes gradually with increasing time period of the fingerprint exposed to the air, and could result in adsorption of the silver powder or the magnetic powder more difficult. That could make the fingerprint fuzzy and indistinguishable.

To resolve the aforesaid problem the smoke fumigation method and Ninhydrin method were developed that do not need direct contact with the fingerprint. The smoke fumigation method adopts a principle of placing an intended test object contained the fingerprint in a closed container, then fill the closed container with a cyanoacrylate gas generated by heating a solution contained cyanoacrylate. The cyanoacrylate generates a polymerizing reaction with the protein or lipid of the fingerprint to form a white substance that can reveal the fingerprint to make it visible. For instance, Taiwan Patent No. M348634 entitled “Automatic smoke fumigation apparatus” which fills first a closed space with cyanoacrylate gas through a smoke fumigation unit, then draws the polymerized exhaust gas through a pumping unit to a filter unit for filtering to reduce toxicity. Because the cyanoacrylate is toxic in nature, it is not advisable in practice even though the toxicity is reduced by filtering. Moreover, the closed container is limited in capacity, hence the size of the intended test object also is limited, and cannot be used for the intended test object of a greater size.

The Ninhydrin method adopts an approach of submerging the intended test object in a trace evidence developing solution. The trace evidence developing solution has a Ninhyrdin ingredient which has reaction with the protein of the fingerprint to generate a colored substance to reveal the fingerprint and make it visible. The Ninhyrdin is not toxic and can be used in an open container during submerging, hence is more desirable than the smoke fumigation method in practice.

In short, all the aforesaid conventional techniques for development of biological trace evidence still have problems such as tend to destroy the biological trace evidence, limitation of the size on the intended test objects and lack portability of equipment.

### SUMMARY OF THE INVENTION

The primary object of the present invention is to solve the problem of the conventional biological trace evidence developing techniques of easily destroying the biological trace evidence, size limitation of the intended test objects and lack portability of equipment.

To achieve the foregoing object the present invention provides an atomizing apparatus for development of biological trace evidence to reveal a biological trace evidence to become visible. The atomizing apparatus comprises a container which has an opening and a housing space communicating with the opening, a trace evidence developing solution held in the housing space and an atomizer located in the opening. The atomizer includes a piezoelectric vibration element in contact with the trace evidence developing solution. The piezoelectric vibration element can shake the trace evidence developing solution via high frequency vibration to form a plurality of small liquid particles that are discharged outside the container. The small liquid particles of the trace evidence developing solution pass through the opening and spray directly on the biological trace evidence to form direct contact therewith to generate an oxidized reduction reaction to reveal the biological trace evidence and make it visible.

Through the technique set forth above, compared with the conventional techniques, the invention can provide many advantages, notably:

1. By spraying the trace evidence developing solution on an intended test object there is no size limitation of the intended test object, and also does not easily destroy the biological trace evidence.

2. The atomizing apparatus adopted for use on the trace evidence development is compact in size, hence is easy to carry around and improves usability.

3. Spraying frequency and amount can be controlled to precisely dispense on the biological trace evidence, hence also can resolve the problem of wasting the trace evidence developing solution that might otherwise take place.

4. The small liquid particles can reveal the biological trace evidence clearer to enhance resolution for identification of the biological trace evidence.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of a first embodiment of the invention.

FIG. 2 is an exploded view of the first embodiment of the invention.

FIG. 3 is a schematic view of the first embodiment of the invention in a use condition.

FIG. 4 is an exploded view of a second embodiment of the invention.

### DETAILED DESCRIPTION OF THE PREFERRED EMBODIMENTS

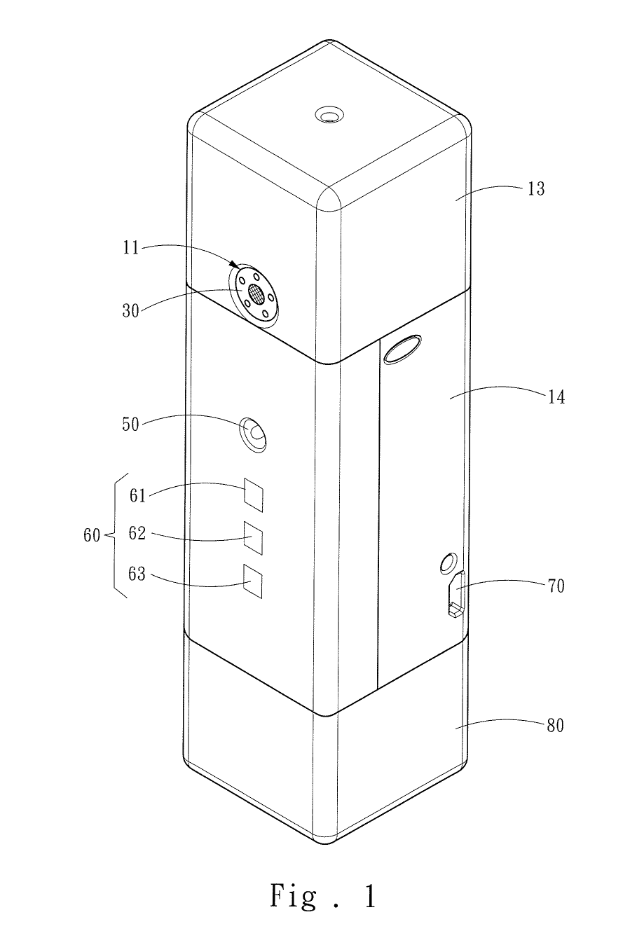

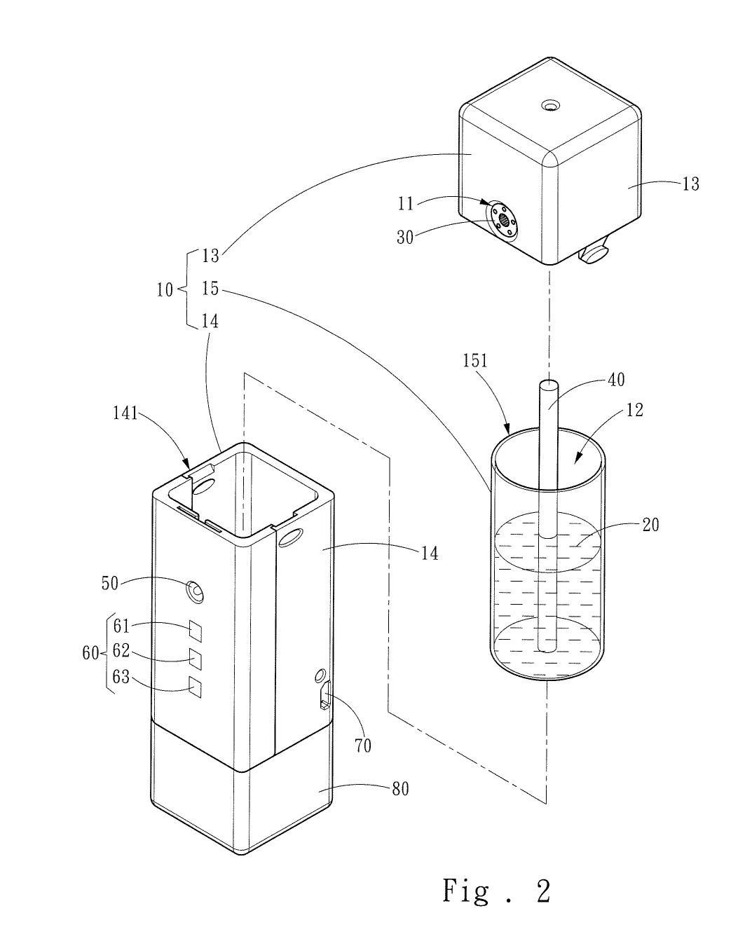

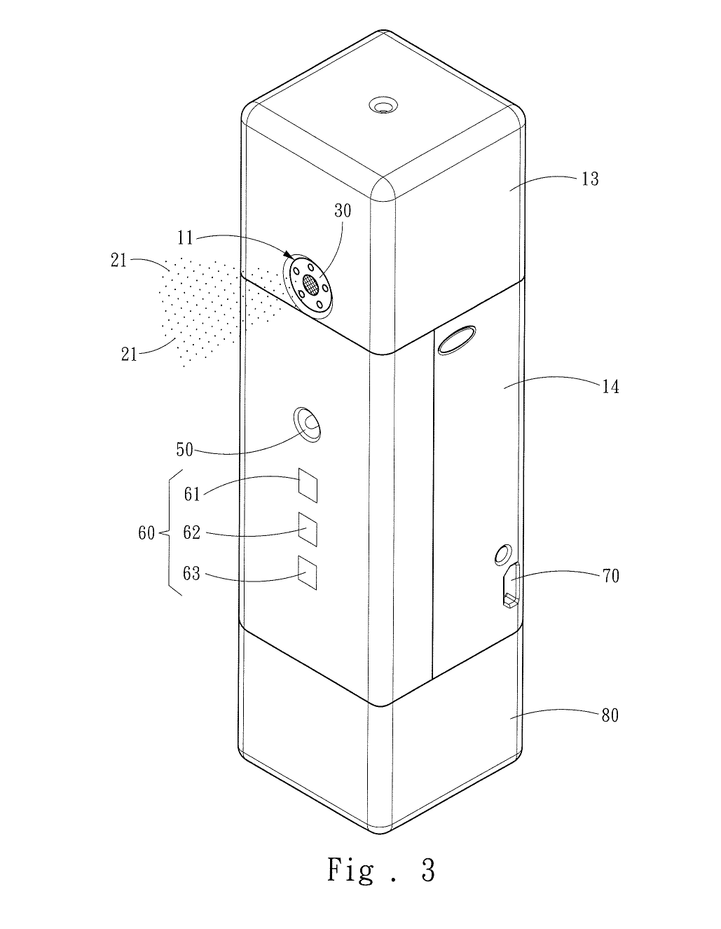

Please refer to FIGS. 1, 2 and 3 for a first embodiment of the invention. The atomizing apparatus for development of biological trace evidence according to the invention comprises a container 10, a trace evidence developing solution 20 and an atomizer 30. The container 10 has an opening 11 and a housing space 12 communicating with the opening 11. The trace evidence developing solution 20 is held in the housing space 12. The atomizer 30 is located in the opening 11 and includes a piezoelectric vibration element in contact with the trace evidence developing solution 20. The piezoelectric vibration element can shake the trace evidence developing solution 20 via high frequency vibration to form a plurality of small liquid particles 21 to be discharged outside the container 10. As shown in FIG. 2, in this embodiment the container 10 further includes a head 13, a casing 14 and an inner housing chamber 15. The opening 11 is located on the head 13. The casing 14 has a first opening 141 to be movably coupled with the head 13. The inner housing chamber 15 is located in the casing 14 and has a second opening 151 communicating with the first opening 141. The housing space 12 is located in the inner housing chamber 15 and communicates with the second opening 151.

The atomizing apparatus further includes a capillary element 40, an illumination element 50, a switch means 60, a Universal Serial Bus (USB) 70 and an electromagnetic unit 80. The capillary element 40 is located in the housing space 12 in contact with the trace evidence developing solution 20 and extended toward the atomizer 30. The illumination element 50 is located at the same side with the opening 11 to provide light projection to make a biological trace evidence to be clearly revealed and seen during developing, or can provide a desired developing effect for a specific trace evidence developing solution 20. The illumination element 50 can be a light source of visible light or ultra violet light. The switch means 60 is located at the same side of the illumination element 50 and includes a first switch 61, a second switch 62 abutting the first switch 61, and a third switch 63 abutting the second switch 62. The first switch 61 is electrically connected to the piezoelectric vibration element to control ON/OFF of the piezoelectric vibration element. The second switch element 62 is electrically connected to the illumination element 50 to control ON/OFF thereof. The third switch 63 is electrically connected to the electromagnetic unit 80 to control ON/OFF thereof. The USB 70 provides a power input source and is located at one side of the casing 14 remote from the head 13 to adsorb a magnetic material such as magnetic powder.

When this embodiment is in use, first, a user sets on the first switch 61 to activate the piezoelectric vibration element; next, the capillary element 40, through capillary effect, transmits the trace evidence developing solution 20 held in the housing space 12 to the atomizer 30 to form the small liquid particles 21 via the high frequency vibration generated by the piezoelectric vibration element; the small liquid particles 21 are ejected through the opening 11 to the biological trace evidence; finally, the trace evidence developing solution 20 and the biological trace evidence generate reaction to form a colored substance; then according to requirement the illumination element 50 can set on through the second switch 62 to provide illumination to make the colored substance visible clearly. Or the electromagnetic unit 80 can be activated through the third switch 63 to adsorb and carry the magnetic powder. The magnetic powder adsorbed by electromagnetic power can be evenly dispensed and adsorbed on the biological trace evidence for developing thereof.

For instance, the biological trace evidence can be a fingerprint. The trace evidence developing solution 20 can include Ninhydrin in the ingredients. Because the fingerprint contains sweat secreted by sweat glands of the skin that is mostly water, but also includes protein, organic compounds, lipid and urea. The Ninhydrin and the protein can generate an oxidized reduction reaction between them to form the colored substance so that the fingerprint can be revealed and seen easily. In addition, the magnetic powder adsorbed by the electromagnetic unit 80 can be adsorbed via static electric charges to the water to clearly reveal the fingerprint. However, the examples mentioned above are not the limitation of the invention. The trace evidence developing solution 20 can also be derivatives of Ninhydrin (such as DFO), silver nitrate, fluorescent ammonia, o-phthalaldehyde, cyanoacrylate, amino-indole, iodine or the like. The biological trace evidence can also be blood stain or other trace evidence.

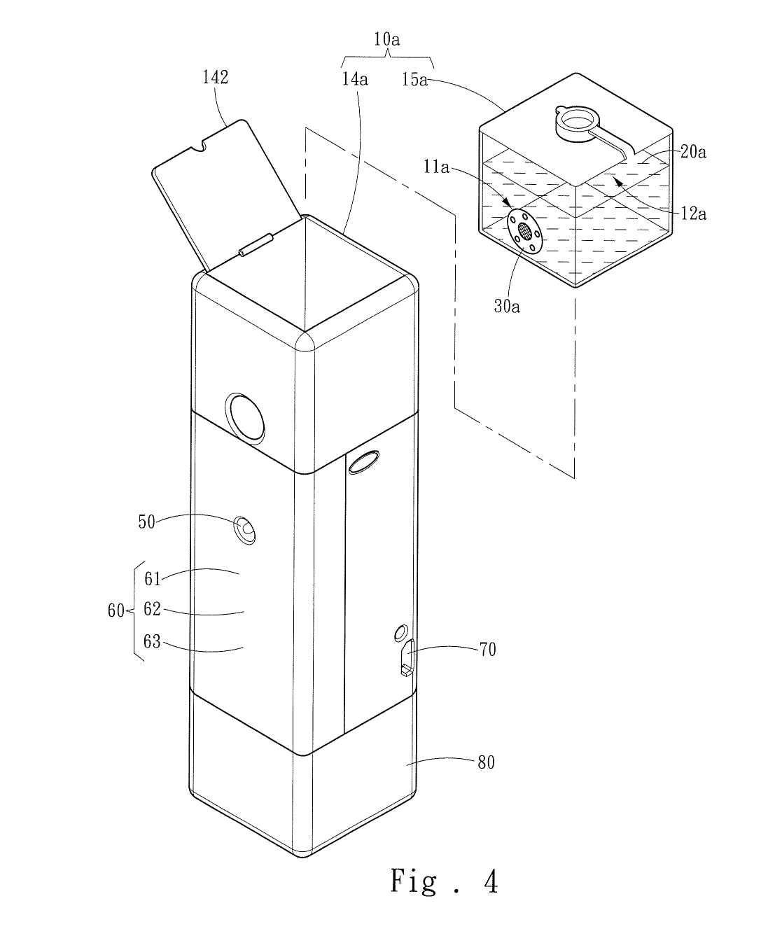

Please refer to FIG. 4 for a second embodiment of the invention. It differs from the first embodiment as follows: the container 10a includes a casing 14a and an inner housing chamber 15a located in the casing 14a. The casing 14a has a flippable cap 142 which can be opened to allow the inner housing chamber 15a to be placed inside. The inner housing chamber 15a includes a housing space 12a to hold the trace evidence developing solution 20a, and also an opening 11a to hold an atomizer 30a. Hence the trace evidence developing solution 20 can be in direct contact with the atomizer 30a to form the small liquid particles (not shown in the drawing) that can be discharged outside the container 10a through the opening 11a.

As a conclusion, the present invention can provide many advantages, notably:

1. By spraying the trace evidence developing solution on an intended test object with the biological trace evidence adsorbed thereon there is no size limitation of the intended test object, and the biological trace evidence also is not destroyed easily

2. The atomizing apparatus adopted for use on the trace evidence development is compact in size, hence is easy to carry around and improves usability.

3. Spraying frequency and amount can be controlled to precisely dispense on the biological trace evidence, hence also can resolve the problem of wasting the trace evidence developing solution that might otherwise take place.

4. The small liquid particles can reveal the biological trace evidence clearer to enhance resolution and accuracy for identification of the biological trace evidence.

5. Through the illumination element visibility and clearness of the biological trace evidence can be enhanced to clearly reveal the biological trace evidence.

6. Through the electromagnetic unit the magnetic powder can be adsorbed and carried to clearly reveal the biological trace evidence.

In short, the present invention provides significant improvements over the conventional techniques.

While the preferred embodiments of the invention have been set forth for the purpose of disclosure, they are not the limitation of the invention, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An atomizing apparatus for development of biological trace evidence to develop a biological trace evidence to be revealed and visible, comprising:
a container including an opening and a housing space communicating with the opening;
a trace evidence developing solution held in the housing space; and
an atomizer located in the opening and included a piezoelectric vibration element which is in contact with the trace evidence developing solution and shakes the trace evidence developing solution through high frequency vibration to form a plurality of small liquid particles to be discharged outside the container;
wherein the small liquid particles are directly sprayed on the biological trace evidence through the opening to generate an oxidized reduction reaction therewith to reveal the biological trace evidence in a visible fashion.

2. The atomizing apparatus of claim 1, wherein the container includes a head, a casing and an inner housing chamber, the opening being located on the head, the casing including a first opening which is movably coupled with the head, the inner housing chamber being located in the casing and including a second opening communicating with the first opening, the housing space being located in the inner housing chamber and communicating with the second opening.

3. The atomizing apparatus of claim 1 further including a capillary element which is located in the housing space in contact with the trace evidence developing solution and extended toward the atomizer to transmit the trace evidence developing solution through capillary effect to the piezoelectric vibration element.

4. The atomizing apparatus of claim 1 further including an illumination element located on an outer side of the container same as the opening.

5. The atomizing apparatus of claim 1, wherein the trace evidence developing solution is selected from the group consisting of Ninhydrin, derivatives of Ninhydrin, silver nitrate, fluorescent ammonia, o-phthalaldehyde, cyanoacrylate, amino-indole and iodine.

6. The atomizing apparatus of claim 1 further including an electromagnetic unit located at one side of the casing remote from the head.
